(19) United States
(12) Patent Application Publication
(10) Pub. No.: US 2001/0020164 A1
(43) Pub. Date: Sep. 6, 2001

(54) Apparatus for delivering radiation energy

(75) Inventors: Stephanos Papademetriou, Sunnyvale, CA (US); Victor C. Esch, San Francisco, CA (US); Miquel M.L. Praca, San Francisco, CA (US); Alan S. Crarer, San Francisco, CA (US)

Correspondence Address: K. ALISON de RUNTZ, C/O SKJERVEN MORRILL MacPHERSON L.L.P., 25 METRO DRIVE, SUITE 700, SAN JOSE, CA, 95110, US

(73) Assignee: Endo Vasix, Inc.

(21) Appl. No.: 09/742,899

(22) Filed: Dec. 20, 2000

Related U.S. Application Data
(62) Division of application No. 09/113,700, filed on Jul. 10, 1998.

(51) Int. Cl. A61B 18/18

(52) U.S. Cl. 606/7

(57) ABSTRACT

An apparatus and method for accurately and reproducibly locating a planar array of optical fibers in space are disclosed. A single acousto-optic modulator is used to accurately and substantially-simultaneously deflect multiple beams of different-wavelength radiation energy to each of said accurately-positioned optical fibers. Both aspects of the invention can be used in an apparatus and method for removing occlusions from vessels, as previously disclosed.

### BACKGROUND OF THE INVENTION

This invention relates generally to the removal of a partial or total occlusion from a body lumen, more specifically to delivering pulses of radiation energy to the body lumen via optical fiber media to generate pressure waves that destroy the occlusion, and even more particularly to the manner of delivering radiation energy to the optical fiber media. The term “clot” is used herein to refer to a thrombus, embolus or some other total or partial occlusion of a vessel.

The technology underlying the present invention is set forth in U.S. patent application Ser. No. 08/955,858, entitled “PhotoAcoustic Removal of Occlusions From Blood Vessels,” filed on Oct. 21, 1997, the entirety of which is herein incorporated by reference.

It is an object of the present invention to improve the delivery of radiation energy to optical fiber media used in opening totally or partially occluded blood vessels.

It is a further object of the present invention to provide an apparatus and technique for accurately and reproducibly positioning one or more optical fibers in space to receive one or more free radiation beams.

It is another object of the present invention to provide a technique and apparatus for essentially-simultaneous delivery of multiple beams of different-wavelength radiation into an optical fiber using a single Acousto-Optic Modulator.

It is another object of the present invention to provide a practical instrument and system to perform these functions.

### SUMMARY OF THE INVENTION

These and other objects are accomplished by the various aspects of the present invention, wherein, briefly and generally, free beam(s) of radiation energy are deflected into one or more optical fibers. Use of an Acousto-Optic Modulator (“AOM”), for example, to deflect the radiation beam(s) requires precise positioning of the optical fibers in space, so that the AOM can accurately and reproducibly deliver the radiation beams to each fiber once the apparatus has been calibrated. Accurate positioning is accomplished with an improved apparatus comprising twin opposed towers and two or more locating pins and a biasing means for accurately and reproducibly positioning in space a cassette containing the optical fibers. A further aspect of the present invention is to use a single AOM to scan two or more different-wavelength radiation beams to the same location in space, such as the tip of an optical fiber, by temporally interspersing the beams.

Additional objects, features and advantages of the various aspects of the present invention will be better understood from the following description of its preferred embodiments, which description should be taken in conjunction with the accompanying drawings.

For further details about the techniques and apparatus used to practice the present invention in the context of removing occlusions from blood vessels, the reader is directed to U.S. patent application Ser. No. 08/955,858.

### BRIEF DESCRIPTION OF THE DRAWINGS

FIG. 1 is an electro-optical diagram of an embodiment of the present invention;

FIGS. 2A, 2B, and 2E are detailed drawings of a connector apparatus used to mount the optical fibers; FIG. 2F is depicts Section A-A of FIG. 2A (with the optical fibers omitted for clarity); FIG. 2C is a drawing of an alignment apparatus to accurately mount the optical fiber connector; and FIG. 2D is a side view of a portion of the apparatus shown in FIG. 2C;

FIG. 3 is a timing diagram showing how the various laser beams of FIG. 1 can be scanned into the fiber optic delivery system;

FIG. 4 is an electronic circuit block diagram of the system control of the embodiment of FIG. 1; and

FIG. 5 is a timing diagram showing various signals of the system control circuit of FIG. 4.

FIG. 6 shows a telecentric arrangement of an embodiment of the present invention.

### DESCRIPTION OF THE PREFERRED EMBODIMENTS

In the apparatus disclosed in U.S. patent application Ser. No. 08/955,858, radiation energy is scanned into the various optical fibers of the connector using a galvanometer and mirror arrangement. Movement of the energy beam from one fiber to another using this arrangement necessarily requires some time for the mirror to mechanically adjust, during which none of the optical fibers is able to receive energy. It will usually be preferable to reduce or eliminate this gap in delivering radiation pulses to the fibers. This can be done by substituting an AOM for the galvanometer/mirror arrangement to controllably scan a radiation beam, such as a laser beam, across the ends of one or more optical fibers.

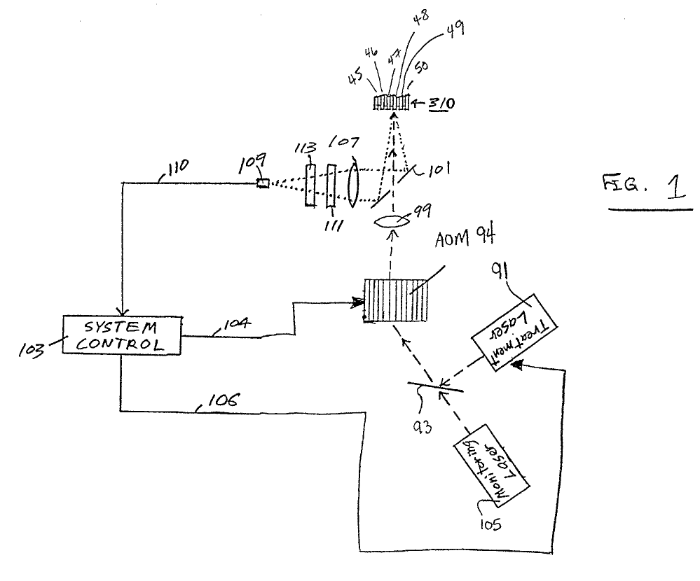

In more detail, FIG. 1, which parallels FIG. 10 of U.S. patent application Ser. No. 08/955,858, depicts an arrangement within the scope of the present invention. As shown in FIG. 1, a treatment radiation source 91, such as a laser or equivalent energy source, and preferably the Q-switched, frequency doubled Nd:YAG laser mentioned in the '858 application, emits radiation pulses of a fixed frequency set to correspond to a desired pulse repetition rate. An input control signal 104 effectively turns the laser 91 on and off by controlling the AOM to cause the beams either to impinge on an optical fiber or to be “blanked” by delivery to a heat sink. The pulses are reflected from a dichroic mirror 93, through AOM 94, and through an optical system 99 that focuses the laser output beam through an aperture of a mirror 101 onto the optical fiber connector 310. This beam is scanned in sequence across a line of the individual fibers 45-50 by the AOM 94 in response to the control signal 104 from a controller 103. The AOM 94 preferably holds the beam on a single optical fiber for enough time to direct a burst of a given number of one to many pulses into that one fiber before moving the beam to another fiber. A signal 104 supplies the correct radio frequency to the transducer of the AOM, depending upon which optical fiber is to receive the output pulses of the laser 91.

Each of the optical fibers 45-50 should be precisely positioned in space relative to the AOM so that the AOM can accurately deliver the treatment laser beam to each fiber. This accurate positioning can be achieved by positioning the fibers in a known relationship in connector 310 and then accurately positioning the connector in space. An AOM typically deflects an incoming light beam so that the incoming and the deflected beams are coplanar—e.g, so that both beams lie in a simple X-Z plane with a constant Y-component. Thus, it is preferable to arrange the optical fiber tip portions in a single plane, as in connector 310, so that the planar fiber tip array can be made to coincide with the AOM's X-Z operating plane by orienting the planar fiber array at the desired Y height. Since the fibers are arrayed in the connector 310 at fixed, known distances from one another, the AOM controller can be programmed to cause the AOM to deliver the laser beam accurately to any desired fiber once the apparatus has been calibrated.

Each optical fiber may be only 50 microns or less in diameter, although fibers of 200 micron diameter of less are adequate. As the diameter of each optical fiber approaches the diameter of the laser beam being focused on the end of each fiber, vertical (Y-) positioning tolerance of the fiber array can approach the order of microns. While one of ordinary skill in the art would recognize that a standard X-Y-Z table with micro-adjusters could be used to accurately vertically locate the fiber array in space so that the AOM's deflected beam is aligned with the first fiber, adjusting the table through trial and error to achieve alignment has proven time-consuming and difficult to reproduce, driving the need for a better alignment mechanism.

Moreover, for a disposable radiation-energy delivery system, designed (e.g.) for single-use only (as is typical in the medical environment), the laser system desirably will include an apparatus for quickly, reproducibly and accurately locating the optical fiber tips in the same position in space relative to the AOM for every disposable, regardless of the change in connectors, so that the AOM can accurately locate the fibers during subsequent operations with a replacement delivery system.

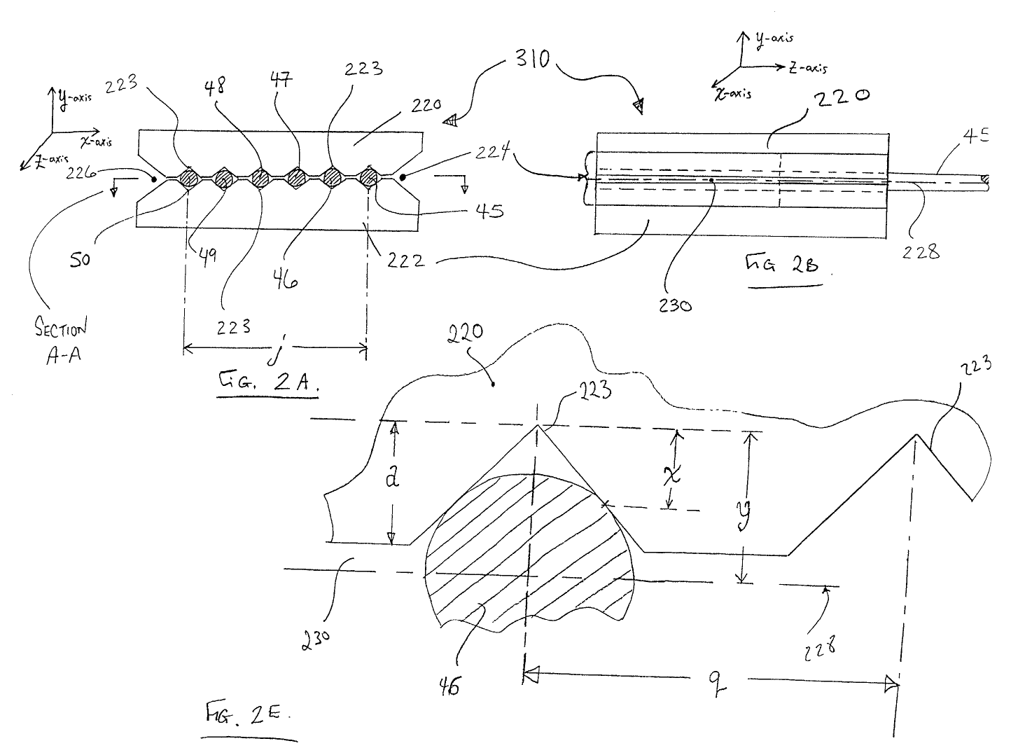

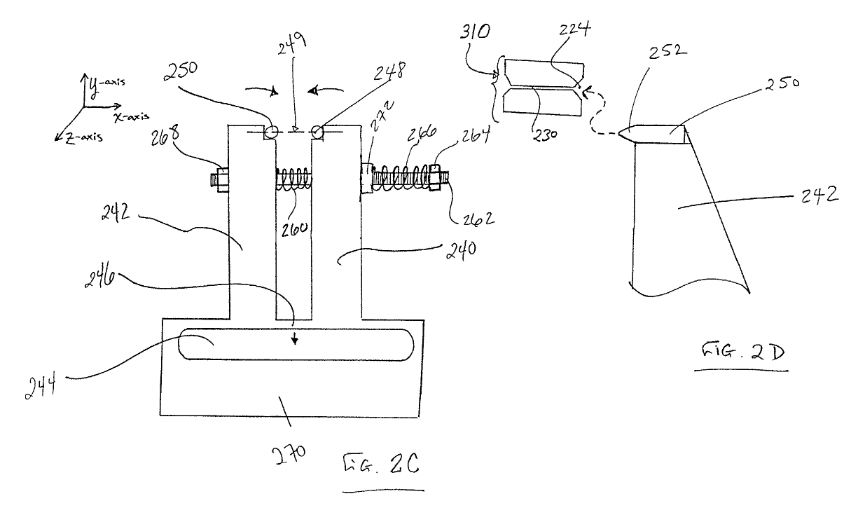

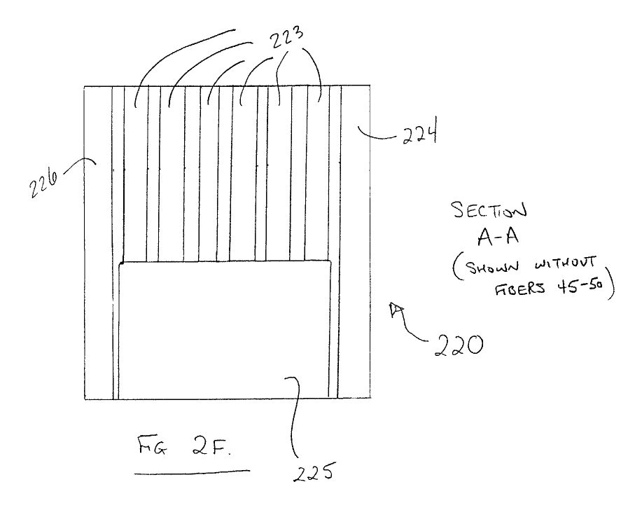

One embodiment of such an alignment arrangement includes the connector 310 shown in FIGS. 2A, 2B and 2E, and the alignment apparatus shown in FIGS. 2C and 2D. Connector 310 of FIGS. 2A and 2B consists of two opposed plates, 220 and 222, preferably of silicon. Each plate has a number of lithographically-etched grooves 223 corresponding to the desired number of optical fibers. Lithographical etching of single crystal silicon is well-known in the art and will not be further described herein. Six grooves 223 are shown for illustrative purposes, one for each of optical fibers 45-50. The silicon plates 220 and 222 are matched and glued as shown, such that the grooves 223 accurately oppose one another and anchor each optical fiber in place, so that the center of each fiber corresponds with the center line 228 of connector 310.

As long as a gap 230 exists between the two sandwiched silicon plates 220 and 222—i.e., as long as the etched depths d of the grooves of each opposed plate, shown in greater detail in FIG. 2E, are substantially equal to one another and are less than y and preferably greater than x (where x is the perpendicular distance between the vertex of the etched groove 223 and the point at which the side of the groove tangentially contacts the circular optical fiber)— the center line of the fibers is assured of aligning with the center line 228 of the connector. The x and y values can be calculated using the trigonometric relationship between the angle of silicon etch and the diameter of the optical fiber. The horizontal distance q between the vertices of adjacent grooves 223 is controlled during etching to within 1 micron so that when the fibers are locked in place between the sandwiched plates, the location of the center of each optical fiber along center line 228 is precisely known.

When sandwiching the fibers between the silicon plates, each optical fiber tip is aligned approximately with the edge of the two silicon plates. Once the fibers are mounted, and the plates are bonded together, and the proximal end of the silicon sandwich is preferably polished to produce optically-clear fiber tips capable of accepting laser light with minimal interference.

As part of the lithographic etching process mentioned above for etching grooves 223, silicon plates 220 and 222 are further lithographically-etched to create portions corresponding to alignment grooves 224 and 226. Alignment grooves 224 and 226 are proportionately much deeper than grooves 223. However, as long as the etch depth of each corresponding alignment groove portion is substantially the same, the center-line of grooves 224 and 226 will correspond to the center-line 228 of the connector and thus of the arrayed fibers 45-50. Moreover, as long as silicon plates 220 and 222 are etched from the same symmetric etching mask, the alignment of the fibers and the alignment grooves can be assured within a very few microns.

FIG. 2F depicts a well 225 preferably etched in each connector plate 220 and 222 to approximately the same depth as each alignment groove 224 and 226. This well relieves stress on the optical fibers at the distal end of the connector, and helps to catch excess glue from grooves 223.

As shown in FIG. 2C depicting the alignment apparatus, two parallel vertical towers 240 and 242, each preferably having a width narrower than a depth shorter than a height, are positioned with broader sides opposing. Both towers are mounted on a rigid base plate 246 that is thin enough to flex slightly to permit the tops of the towers to approach one another when biased together. A unitary construction is preferred, although the towers could be fixedly attached to the baseplate by an appropriate means.

Material for the unitary apparatus may be stainless steel or aluminum, although any material providing the required rigidity and flexibility may be used. Preferably, the alignment apparatus is composed of the same material as the remainder of the laser apparatus, so that any expansion or contraction of the entire apparatus due to changes in the ambient temperature of the operating environment will result in approximately equal deformation across all components, thereby maintaining alignment. For a unitary stainless steel construction, a baseplate thickness of about 1 mm has proven adequate for towers of about 8 mm thick and 30 mm high. Such a base plate 246 can be created by drilling a hole 244 out of a larger block of material 270. The baseplate may also be drilled so that less than the entire footprint of each tower actually contacts the baseplate, thereby increasing the capability of the towers to flex towards one another. For an overall tower footprint depth of 40 mm, removal of approximately the middle 20 mm of material so that each tower is supported only by two 10 mm-deep feet has proven adequate.

Each tower 240 and 242 has a notch to seat dowels 248 and 250, respectively, as shown in FIG. 2C. Dowels 248 and 250 may be held in place by locking plates (not shown) or some other suitable means such as glue. Towers 240 and 242 are spaced sufficiently far apart so that the gap between the interior edges of dowels 248 and 250 is wide enough to comfortably horizontally seat connector 310. As shown in FIG. 2D, each dowel has a beveled end 252. Connector 310 is seated between towers 240 and 242 by sliding grooves 224 and 226 onto the beveled ends of dowels 250 and 252, respectively. Alignment grooves thus permit the connector to “self-align” to the exact known height of the centerline 249 of the dowels such that centerline 249 and centerline 228 substantially coincide. Given the diameter of the dowel pins (which is a constant in each given assembly), the alignment grooves of the silicon cassette connector will spread the towers slightly outward to a distance between the dowel pin centerline that is repeatable within a few microns for different disposable connectors.

When the positioning apparatus is first fixed in place relative to the AOM, the height of the center line 249 of dowels 248 and 250—and thus the ultimate location of the planar array of optical fibers once the connector 310 is seated—is targeted by the adjustable optics to be within approximately +/−2 microns of the X-Z operating plane of the AOM Once the fiber array is accurately targeted, the fiber positions are reproducible from connector to connector, since the centerline of the dowels remains fixed.

Preferably a shutter (not shown) is included in the positioning apparatus. When the connector 310 is seated into position between dowels 248 and 250, the shutter is opened and locked into place, thereby permitting laser light to enter the proximal ends of the optical fibers. When the connector is removed from the apparatus, the shutter drops down into a position that blocks any further laser light from entering the assembly until another connector is seated. The shutter may also be constructed to lock the connector into position between the dowels so that the X-Z array of fibers occupies a particular location along the Z-axis. In this manner, the arrays of optical fibers in different connectors would reproducibly occupy the same Z-axis location relative to the AOM— i.e., the same distance from the AOM— and thus ease positioning of the fiber array in the focal plane of the lens 99. Finally, since the shutter blocks the laser beams during connector replacement, the shutter can be marked in such a way that it can be used to verify positioning of the laser beams during selected down-times.

A threaded rod 262 is passed through both towers 240 and 242. A structure, such as a nut 268, is place on one end to prevent the rod from pulling out of tower 242. Washer 272, spring 266, and nut 264 are arranged on the other end of the threaded bar 262 such that when the nut is tightened, the spring 266 biases towers 240 and 242 equally slightly towards each other as shown by the arrows, thereby clamping the seated connector accurately in place between dowels. Flexing in the towers due to this biasing is typically no more than 20 microns. Springs 260 and 266 are chosen for their geometry and stiffness to provide a bias to the geometry of the towers such that nut 262 can provide fine adjustment for the distance between dowels 248 and 250 when tightened or relaxed. The springs allow for adjustment without significantly affecting the overall stiffness of the tower system, as well as providing a very fine resolution of the adjustment.

Returning to FIG. 1, a second laser 105 is provided to monitor the existence of a bubble as previously described in the '858 application. Monitoring laser 105 can be a simple continuous wave (cw) laser with an output within the visible portion of the radiation spectrum. Its output beam is chosen to have a sufficiently different wavelength from that of the treatment laser 91 to enable the two laser beams to be optically separable from each other. A helium-neon laser is appropriate, as is a simpler diode laser with an appropriate wavelength and a maximum output of 10 milliW, for example.

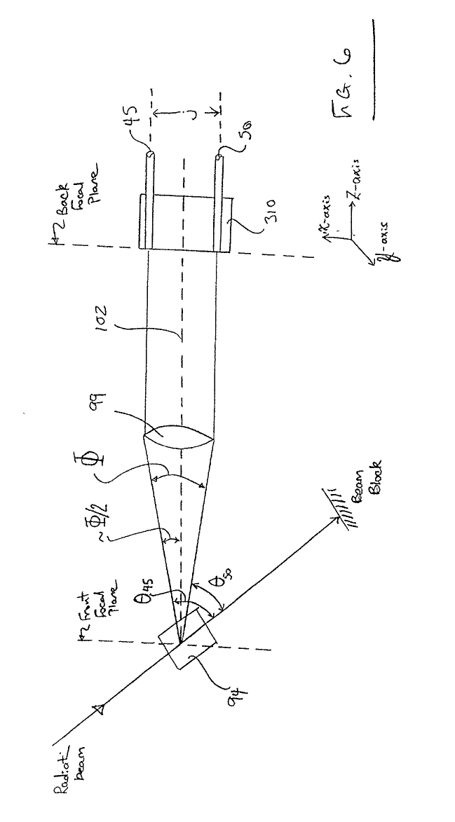

It was previously believed by others that if an AOM for scanning the treatment beam were to replace the galvanometer/mirror arrangement of the '858 application, another AOM would be required for scanning the monitoring beam. It was thought that another AOM would be needed for the monitoring beam in addition to the AOM managing the treatment beam because a single AOM cannot simultaneously scan two beams having different wavelengths to the same point in space, such as the tip of a single optical fiber. The angle (Θ, in radians) to which an AOM deflects an energy beam is a function of the energy's wavelength (80 ):

$$Θ = \frac{λ \cdot f}{v ( T )}$$

where ƒ is the AOM modulation frequency and ν(T) is the speed of sound in the AOM optical crystal material (typically TeO) at temperature T. Thus, for a given modulation frequency, different wavelength energy beams, such as the treatment and monitoring beams, will be deflected at different angles. Two examples of Θ—corresponding to fibers 45 and 50—are shown in FIG. 6.

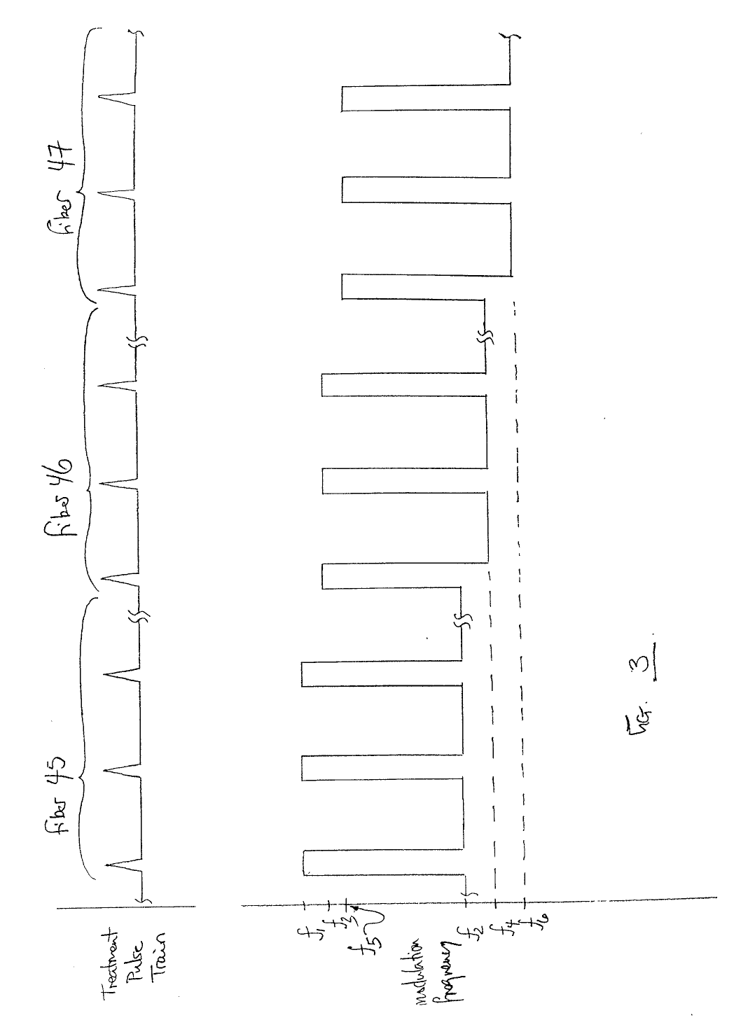

Contrary to the previously-held belief, a single AOM may be used to deflect both the treatment laser beam and the monitoring laser beam in this invention, as shown in FIG. 1. Use of a single AOM is made possible by using a pulsed treatment laser and deflecting the monitoring during the “down-time” between consecutive treatment laser pulses. More specifically, after a first treatment laser pulse and before the next pulse, the modulation frequency of the AOM is first adjusted so that the monitoring laser beam is deflected to the same optical fiber that received the first treatment laser pulse, and is then readjusted so that the next treatment laser pulse will also land on the same optic fiber. This operation is shown in more detail in FIG. 3. The AOM modulation frequency for the single AOM can be temporally adjusted between a pair of frequencies (ƒ1 and ƒ2) to permit the desired number of treatment laser pulses and associated monitoring laser beams, respectively, to pass down a single optical fiber 45 before switching to the next pair of frequencies (ƒ3 and ƒ4) to shift focus to the next fiber 46 to receive energy, and so on. FIG. 3 shows three pulses per fiber, although any number may be used as desired. Likewise, although FIG. 3 illustrates shifting the monitoring beam after every treatment pulse, some other arrangement may be desired.

To operate with a single AOM in this manner, the wavelengths of the treatment and monitoring beams need to be similar enough that the range of modulation frequencies necessary to deflect both laser beams to each of the optical fiber positions falls within the AOM's bandwidth, but yet dissimilar enough that the beams can be combined while remaining substantially free of mutual interference. Satisfactory results were achieved with a treatment laser wavelength of 532 nm, a monitoring beam of 635 nm and an AOM with a bandwidth of about from 50 to 100 MHz.

Consistent with the various parameter ranges described in the related U.S. patent application Ser. No. 08/955,858, which are included herein as a result of the incorporation by reference of the '858 application, satisfactory results using the present inventions to treat vessel occlusions have been achieved using a 5 kHz pulse rate, each pulse having a duration of around 25 ns; a 0.3 duty cycle; and shifting focus to another fiber after delivering a sequence of 3-on/7-off pulses of energy to a first fiber. For the 50/55/65 micron core/clad/jacket optical fibers disclosed in the '858 application, which dimensions are useful to increase flexibility and minimize volume requirements, energy per pulse ranges up to about 400 microJ, with around 200 microJ being preferred, have been used successfully. Average energy delivered to the vessel being treated is preferred to be less than about 0. 5 W, with about 300 milliW being preferred. Refractive index values for the various materials of the optical fibers that result in a numerical aperture in excess of 0.20 are practical, such as a numerical aperture of 0.22 to 0.26, or even 0.29.

Because ƒ is dependent upon the temperature of the AOM, it can be affected by both changes in ambient operating conditions and changes due to the self-heating mechanisms of (a) the laser beam passing through the AOM crystal and (b) the deflection energy delivered to the AOM via the rf transducer. To compensate for possible, uncontrolled temperature variations due to these sources, the AOM's operating temperature is controlled to an artificially high value—e.g., between 45 and 50 degrees Centigrade. This may be achieved by, for example, applying energy to heating elements present in several heat sinks surrounding the AOM, measuring the resulting operating temperature with a thermistor present in a centrally-positioned heat sink, and operating the AOM only after the AOM has reached the desired operating temperature.

In order to minimize energy losses between the AOM and the fibers shown in FIG. 1, the AOM 94, lens 99, and the fiber optic array are preferably arranged in a telecentric system, although a non-telecentric system would still work. In other words, as shown in FIG. 6, the fiber optic array is preferably centered and positioned on the lens' back focal plane and the AOM's point of rotation sits approximately on the intersection of the front focal plane of lens 99 and the centerline 102 of the array of optical fibers. Telecentricity and telecentric systems are known to one of ordinary skill in the art.

Appropriate AOM/lens combinations for a telecentric system are identified as follows, as would be recognized by one of ordinary skill in the art. First, to minimize energy losses, the spot size of the radiation energy delivered to each fiber through lens 99 ideally is less than the fiber's core diameter. Spot size d is proportional to the focal length f of lens 99:

$$d α \frac{f \cdot λ}{D}$$

where λ is the wavelength of the radiation beam, and D (which is typically limited by an AOM's available aperture) is the diameter of the collimated radiation beam delivered to lens 99 from the AOM. Thus, a small focal length produces a desirably small spot size, for example, in the order of 20 microns,

Second, as already mentioned, the AOM must be able to deflect the radiation beam through lens 99 and into each of the fibers in the planar array. In other words, the AOM must be able to deflect the beam between a minimum and a maximum angle of deflection corresponding to the positions of the tips of the two outermost optical fibers 45 and 50 in the array. The angular difference, Φ, between the minimum and maximum angles of deflection (respectively, Θ50 and Θ45) is related to the distance between the centers of the outermost fibers, j, shown in FIG. 2A:

j=2·f·tan {Φ/2{

Generally, as Φ gets smaller, the AOM becomes less expensive and avoids deflection inefficiencies. For a given j, the necessary angular deflection range Φ is minimized by increasing the focal length, which competes with the desire to decrease the focal length f so as to minimize the spot size d. Given j for the optical fiber array, a wavelength λ, and the optical fiber diameter (which the spot width d should not exceed if energy loss is to be minimized, as discussed above), an appropriate lens and AOM combination can be chosen depending on the desired size/cost/availability of the laser/AOM/connector system. Finally, because the AOM's point of rotation is located approximately on the centerline of the fiber array in the telecentric system, Φ is roughly ideally bisected by the array's centerline.

Returning to FIG. 1, after the treatment laser beam is delivered to an optical fiber, it travels down that fiber and into the lumen being treated. The contents of the lumen then absorb the energy pulse and, ideally, a bubble will result. When a bubble forms at the distal end of the optical fiber receiving both of the treatment and monitoring beams, there is a greater reflection of the monitoring beam than when no bubble forms. The intensity of the monitoring beam reflected from a bubble is much different than the amount reflected in the absence of a bubble because of the different refractive indices of water vapor and lumen fluid. The reflected monitoring beam emerges from the proximal end of the optical fiber, is reflected by the mirror 101 and is focused by appropriate optics 107 onto a photodetector 109 which has an electrical output 110. This reflected monitoring beam is passed through a linear polarizer 111 to reject radiation reflected from the proximal end of the optical fiber. A filter 113 is also placed in the path of the reflected monitoring beam in order to prevent reflected radiation from the treatment laser 91 from reaching the photodetector 109.

Information from the photodetector is preferably used to control delivery of the treatment laser. Briefly, returning to FIG. 3, the monitoring beam may be the first beam scanned down an optical fiber while the treatment beam is between pulses. The amount of light detected by photodetector 109 as a result of this initial monitoring beam scan-and-feedback can be considered a baseline DC noise level.

After the AOM frequency is adjusted to the value required to shift the treatment beam to the same fiber, the pulse of treatment radiation is delivered to the fiber and hence to the vessel being treated. The measured reflection of a subsequent monitoring beam detected by photodetector 109 increases over time to reflect the formation of a bubble. After a certain period of time corresponding to the duration of the bubble's existence, the photodetector signal decreases back to the baseline reading, indicating bubble collapse. The baseline DC level for that fiber is backed out of the increasing/decreasing photodetector signal, thereby producing photodetector readings that represent the net increase/decrease in reflection due to bubble formation/collapse. These net values can be amplified so that the data are more accurately distinguishable from one another and can be more easily manipulated. For each set of increasing/decreasing photodetector signals, the “width” of the readings, corresponding to the duration of the bubble's life, is calculated by determining the Full Width Half Max value. The width and amplitude measurements can then be used to control operation of the treatment laser.

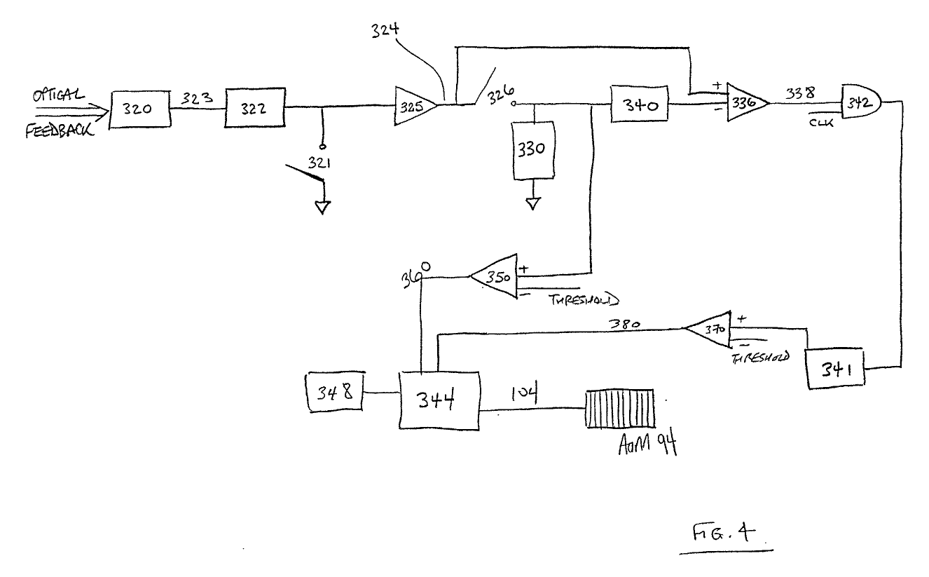

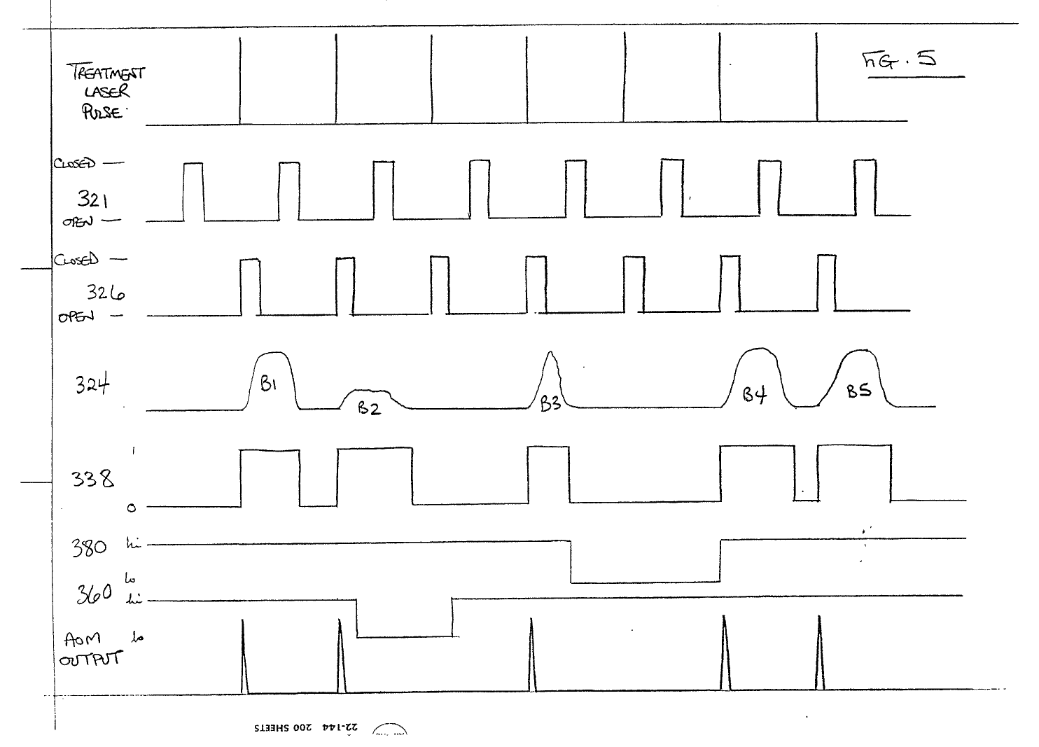

In more detail, a block electronic circuit diagram for a portion of the system control 103 of FIG. 1 is given in FIG. 4, with several of its signals being given in the timing diagram of FIG. 5. The optical signal impinging on photodetector 109 is converted from a current to a voltage signal by circuit 320. Circuit 320 may comprise a photodiode and amplifier. The amplifier should have a sufficiently wide gain bandwidth to produce a risetime in the order of a few microseconds. A million-ohm amplifier has proven adequate for this. If the gain bandwidth of circuit 320 is not wide enough, longer risetimes are produced, which results in distortion of the electro-optical signal.

Next, the baseline DC level from each individual fiber is preferably subtracted from the total voltage signal 323 so that only voltage information representative of the actual bubble is produced and further processed. To accomplish this, switch 321 is closed for a certain time period before delivery of the treatment laser pulse to the fiber that is next to receive the pulse, e.g., fiber 45. With switch 321 closed, a capacitor in circuit 322 charges to the baseline voltage level corresponding to the background DC level of fiber 45. When switch 321 is opened, circuit 322 holds that baseline voltage. Once the treatment laser pulse is triggered for fiber 45, the resulting optical feedback is converted to a voltage 323 by circuit 320, which is then effectively reduced by the baseline voltage held in circuit 322 to produce a voltage 324 leaving buffer amplifier 325. “Bubble” voltage 324 represents only the bubble-induced voltage, since the background DC level has been subtracted. The timing of these events is depicted in FIG. 5.

After a treatment pulse is triggered, and during the 5-10 microsecond delay between treatment pulse delivery and bubble formation, switch 326 is closed. As the resulting bubble develops, reflection of the monitoring laser beam up fiber 45 increases, thereby increasing the value of “bubble” voltage 324. Since switch 326 is closed, a capacitor in circuit 330 will charge as value 324 increases. After a certain period of time, switch 326 is opened, and circuit 330 holds the peak voltage representative of the maximum amplitude of the bubble signal. When switch 326 is to be opened is empirically predetermined based on the dynamics of bubble formation for the particular energy level, ambient environment, pulse duration, and absorption characteristics of the system. The goal is to open switch 326 after the bubble has reached its maximum amplitude. For the parameters described herein, opening switch 326 within 20 to 30 microseconds after the treatment pulse has proven adequate.

Next, the bubble “width” , τ, is measured. Voltage comparator 336 compares the actual “bubble” voltage 324, which is representative of the size of the bubble at a particular instant, with a value representing a certain percentage of the peak voltage stored on circuit 330. The percentage of the peak, calculated by circuit 340, can in theory be any desired portion of the peak. A typical width benchmark is 50% mark, which yields a Full Width Half Max value. As the percentage decreases, however, the risk of affecting the “width” reading with noise increases. The output 338 of voltage comparator 336 will remain high as long as the “bubble” voltage 324, representing the bubble's trailing edge, is greater than the chosen percentage of the peak value. For example, if 50% is used, then the output 338 of comparator 336 will remain high as long as the voltage 324 exceeds half the peak voltage stored in circuit 330. As the bubble decays at the end of fiber 45, the bubble voltage 324 will eventually drop below the 50% peak value, causing the output 338 of comparator 336 to go low.

Comparator output 338 gates clock 342. As long as comparator output 338 remains high, counter 341 counts the number of clock cycles from the clock 342. When the comparator output 338 goes low, the counter 341 stops counting. The counted value stored in counter 341 represents the bubble's width.

The counter can be controlled to count from the time the treatment pulse is first fired down fiber 45, and thus can measure the “width” of the bubble from the time the treatment laser is fired. The system, however, can also operate to determine a different width by triggering the counter 341 to count based on leading edge data of the bubble other than the firing of the treatment pulse. Regardless of how the width is determined, what is important is that the method of measuring bubble width remains consistent.

Comparator 350 compares the value of the peak voltage stored in circuit 330 to a predetermined reference level corresponding to a minimum acceptable amplitude threshold, which value depends upon the bubble formation dynamics, chosen gain in the amplified signal, and the system optics. Comparator 350 provides a binary output 360 that is high if the threshold is exceeded and low if not.

Comparator 370 compares the bubble width data to τmin, an empirically-predetermined value that represents the minimum acceptable width of a bubble that is chosen as an indicator that a sufficiently-viable bubble has been formed. Comparator 370 provides binary output 380 that is high if the threshold is exceeded and low if not τ may fail to exceed τmin when desirable operating conditions have not been achieved, which, for example, may be due to no adequate bubble formation or the optical fiber is impinging the vessel wall, as opposed to forming bubbles in blood. An acceptable τmin depends on an array of factors, including bubble dynamics, system energy and absorption parameters, and the particular optical arrangement used. An acceptable τmin can be determined by overlaying a number of bubbles (324 signals) caused by different pulses of treatment radiation on an oscilloscope and then picking a value above which the bubble's width is deemed to be acceptable. For the parameters disclosed herein, an acceptable τmin might be between about 25-35 microseconds.

Either or both of the measured maximum peak amplitude data (binary output signal 360) and the bubble width data, τ (binary output 380), either of which may also be calculated using software or programmable hardware instead of the logical hardware disclosed, can be used to control the treatment laser. Preferably both are used to control the treatment laser. If the feedback laser control is enabled, and if either (a) τmin exceeds the bubble width data τ, such that output 380 is low, or (b) the minimum acceptable amplitude voltage threshold value exceeds the peak amplitude voltage stored in circuit 330, such that output 360 is low, then counter 348, corresponding to fiber 45, is primed with a value corresponding to the number of subsequent pulses of the treatment laser that are to be suppressed for that fiber. For example, if only one pulse is to be suppressed down fiber 45 if a sufficiently-viable bubble fails to develop, as is depicted in FIG. 5, then counter 348 is primed with the value 1. When the treatment laser 91 is next to be fired down fiber 45, the controller 344 first checks the fiber's corresponding counter 348. If the counter contains a value greater than 0, the AOM controller decrements counter 348 by 1 and causes the AOM to “blank” or “zero order” the pulse into a heat sink, such as a block of metal. The net effect is that no treatment pulse is delivered to fiber 45 because that fiber previously failed to produce an acceptable bubble. After the appropriate number of subsequent pulses have been blanked— just one in this example—the fiber counter 348 reaches 0, and the next treatment pulse is permitted to travel down the fiber, to begin the whole process again. Suppressing treatment pulses in this manner is consistent with the goals of the underlying invention of preventing damage to the vessel wall and minimizing the amount of heat delivered to the vessel.

Alternatively, if both the bubble width data τ exceeds τmin, and the amplitude value stored in circuit 330 exceeds the minimum acceptable amplitude threshold, such that both outputs 360 and 380 are high, then the counter 348 is not incremented, which in turn permits the next treatment laser pulse to fire down fiber 45 without being suppressed.

While this control scheme only compares τ to a minimum value that it must exceed, it may also be compared to a τmax. If τ exceeds τmax, then it is believed that the tip of the optical fiber is firing and creating a bubble in blood, for example, as opposed to in clot. This information may be useful to a surgeon using the apparatus. Like τmin, τmax is a function of a variety of variables and thus should be determined empirically for the particular system.

In addition to controlling the laser, the various logical states based on the amplitude and bubble width data can also trigger an audio signal to aurally inform the user of which of the several operating states— e.g., no bubble v. bubble in clot v. bubble in blood— is then occurring.

FIG. 5 shows the relative temporal relationships between the treatment pulses of radiation delivered to fiber 45; the relative positions of switches 321 and 326; signal 324 representing the bubble formation data; the high-low output of comparator 336; the binary output 360 of circuit 350 resulting from comparing the peak amplitude to the amplitude threshold value; the binary output 380 of circuit 370 resulting from comparing the bubble width data to the width threshold value; and the resulting AOM output pulse to fiber 45. As shown in FIG. 5, bubbles B1, B4 and B5 have both acceptable amplitude and width. Bubble B2 has an insufficient amplitude, causing the subsequent treatment pulse to be suppressed. Bubble B3 has an insufficient width, which also causes the subsequent treatment pulse to be suppressed.

While the above describes checking for the existence or nonexistence of a bubble after each treatment pulse, this is only one of many specific arrangements and timing schemes that can be implemented. For example, the existence or nonexistence of a bubble can be determined after each burst of treatment laser pulses. Further, for example, the lack of the detection of a bubble can be used to disable that fiber for more than one cycle, and perhaps for the entire treatment. In the case where only one or a very few pulses are contained in each burst, the detection of the absence of a bubble at the end of one fiber can be used to disable the system from sending treatment radiation pulses down that fiber for a certain number of cycles and then trying again.

Although the various aspects of the present invention have been described with respect to their preferred embodiments, it will be understood that the invention is entitled to protection within the full scope of the appended claims.

It is claimed:

1. A method of scanning multiple radiation energy beams, comprising:
providing a first frequency to an acousto-optic modulator,
transmitting a first beam having a first wavelength to said acousto-optic modulator such that said first beam is deflected to a first location,
providing a second frequency to said acousto-optic modulator, and
transmitting a second beam having a second wavelength to said acousto-optic modulator such that said second beam is deflected to said first location.

2. The method of claim 1 or 5, wherein said first and second beams are simultaneously transmitted to said acousto-optic modulator.

3. The method of claim I wherein said first beam is pulsed.

4. The method of claim 3 wherein the second beam is continuous wave.

5. The method of claim 3, wherein the frequency of said acousto-optic modulator is switched from said first frequency to said second frequency such that said second beam is transmitted to said first location between consecutive pulses of said first beam.

6. The method of claim 1, wherein said first an d second radiation beams are laser beams.

7. The method of claim 1 or 5, further comprising repeating varying the frequency of said acousto-optic modulator such that at least multiple said first beams impinge on said first location.

8. The method of claim 7, wherein the frequencies are repeatedly varied between said first and second frequencies such that a plurality of sets of first and second beams impinge on said first location.

9. The method of claim 7, wherein fewer second beams than first beams impinge upon said first location.

10. The method of claim 1, further comprising
providing a third frequency to an acousto-optic modulator,
transmitting said first beam to said acousto-optic modulator such that said first beam is deflected to a second location,
providing a fourth frequency to said acousto-optic modulator, and
transmitting said second beam to said acousto-optic modulator such that said second beam is deflected to said second location.

11. The method of claim 10, further comprising repeating varying the frequency of said acousto-optic modulator between said first and second frequencies such that multiple sets of said first and second beams impinge on said first location, before said frequency is switched to said third and fourth frequencies.

12. The method of claim 11, wherein fewer than ten pulses of said first beam impinge upon said first location before said frequency is varied between said third and fourth frequencies.

13. The method of claims 1 or 5 wherein said first beam wavelength is about 532 nm and said second beam wavelength is about 635 nm.

14. The method of claim 1 or 5, wherein said first location comprises an optical fiber, such that said first and second beams consecutively enter said optical fiber.

15. The method of claim 14 wherein said first and second radiation beams are delivered via said optical fiber to a body lumen having contents comprising an at least partial occlusion, said method further comprising said first beam interacting with the contents of said body lumen and at least partially removing said occlusion to improve flow through said lumen.

16. The method of claim 15, wherein said interaction between said first beam and said lumen contents comprises generating a shock wave to impinge upon said occlusion.

17. The method of claim 16, wherein said interaction further comprises generating a bubble to impart further stress on a portion of said occlusion.

18. The method of claim 15, wherein said second beam is used to monitor the interaction of said first beam with the contents of said body lumen.

19. The method of claim 17, wherein said second beam is used to monitor the generation of said bubble.

20. The method of claim 19, wherein the first beam is discontinued when the second beam indicates that the first beam did not generate a bubble.

21. An apparatus for accurately delivering a free radiation energy beam to an object located across a void, comprising:
a source of radiation, said source providing a beam of radiation energy having a proximal and a free distal end;
means for scanning said radiation beam within a predetermined plane; and
means for positioning said object substantially within said plane, such that said means for scanning is able to scan said free distal end of said radiation beam to said object.

22. An apparatus for accurately delivering a free radiation beam, comprising:
a source of radiation providing a beam of radiation having a proximal and a free distal end;
an acousto-optic modulator for scanning said radiation beam within a predetermined plane;
a connector housing proximal ends of a substantially planar array of optical fibers, said array positioned remotely from said proximal end of said energy beam; and
a positioning apparatus comprising two towers, said connector positioned between said towers such that said planar fiber array is substantially coplanar with said predetermined plane and such that said modulator can scan said distal end of said beam to each of said optical fibers.

23. The apparatus of claim 22 wherein said beam of radiation comprises multiple pulses of laser radiation.

24. The apparatus of claim 22, wherein distal ends of said optical fibers are mounted in a catheter for introduction into the human vasculature.

25. The apparatus of claim 24, wherein said optical fibers are for delivering radiation energy into cerebral vasculature to remove a portion of an at least partial occlusion from a vessel.

26. An apparatus for accurately positioning an object in space, comprising:
two rigid towers, each tower having a base, an opposed wall, and a portion remote from the base;
a baseplate, said tower bases attached to said baseplate such that said opposed walls face each other; and
means for biasing said towers towards each other to grip an object positioned between said opposed walls of said remote portions of said towers.

27. The apparatus of claim 26, wherein said object comprises at least one optical fiber, said fiber positioned to receive a free beam of radiation energy.

28. The apparatus of claim 26, wherein an axis of said object is substantially perpendicular to said remote portions of said towers.

29. The apparatus of claim 26, wherein said object comprises a planar array of optical fibers, wherein said object is oriented such that said planar fiber array is substantially coplanar with a plane in which an acousto-optic modulator is able to deflect a free beam of radiation into each of said fibers.

30. The apparatus of claim 26 or 29, wherein each opposed tower wall comprises a rod-like structure, wherein said object is positioned between said substantially parallel rod-like structures.

31. The apparatus of claim 29, wherein each opposed tower wall comprises a rod-like structure having a longitudinal axis substantially coplanar with said plane, wherein said object is positioned between said rod-like structures such that said fiber array is substantially coplanar with said plane.

32. The apparatus of claim 26, wherein said baseplate flexes slightly to permit the opposed walls of said remote portions of said towers to move toward or away from one another.

33. The apparatus of claim 26, wherein said towers are integrally attached to said baseplate.

34. The apparatus of claim 29, wherein said radiation beam is to be delivered via said optical fibers to a mammalian body lumen for use in removing a portion of a total or partial occlusion from said lumen.

35. An apparatus for accurately positioning a plurality of optical fibers within a predetermined plane, comprising:
a connector comprising two plates, each plate having a plurality of grooves in a surface, said plates integrally connected so that said grooved surfaces face each other and said pluralities of grooves align to form a plurality of positioning channels in a predetermined arrangement;
a plurality of optical fibers, each fiber having a proximal end positioned within a corresponding one of said channels, such that the proximal ends of said fibers form a substantially planar fiber array;
said connector further having at least one alignment groove positioned in each of two opposite surfaces of said connector, said alignment grooves for positioning said connector between two opposed rod-like structures so that said fiber array is substantially coplanar with said predetermined plane.

36. The apparatus of claim 35, wherein each of said two opposed plates comprises silicon, and wherein said grooves in each plate are lithographically-etched.

37. The apparatus of claim 35, wherein each of said grooves corresponding to a positioning channel has a substantially equal depth and wherein said opposed plates do not directly contact one another when said fibers are positioned in said channels to form said planar fiber array.

38. The apparatus of claim 35, wherein said two rod-like structures are substantially parallel, and each of said structures has a longitudinal axis substantially coplanar with said predetermined plane.

39. The apparatus of claim 35, wherein each of said alignment grooves has a centerline substantially coplanar with the plane of said fiber array.

40. The apparatus of claim 35 wherein said channels are substantially parallel and evenly spaced.

41. The apparatus of claim 35, wherein said predetermined plane is substantially coplanar with a plane in which an acousto-optic modulator is able to deflect a free beam of radiation into each of said optical fibers.

42. The apparatus of claim 41, wherein said radiation beam has a radiation source comprising a laser.

43. The apparatus of claim 41, in which said radiation beam is to be delivered via said optical fibers to a mammalian body lumen for use in removing a portion of a total or partial occlusion from said lumen.

44. The apparatus of claim 35, wherein said plates are connected with glue and said glue and said channels cooperate to hold said fibers immovably in said planar fiber array.

45. An apparatus for accurately positioning an array of optical fibers, comprising:
a connector having a plurality of channels,
a plurality of optical fibers, each fiber having a proximal end positioned within a corresponding one of said channels such that the proximal ends of said fibers form a substantially planar fiber array;
said connector further having at pair of alignment grooves for positioning said connector between two opposed, substantially parallel rod-like structures, said rod-like structures and said alignment grooves interacting so that said fiber array is substantially coplanar with a predetermined plane.

46. The apparatus of claim 45, wherein each of said channels comprises lithographically-etched silicon.

47. The apparatus of claim 45, wherein said two rod-like structures are substantially parallel, and each of said structures has a longitudinal axis substantially coplanar with said predetermined plane.

48. The apparatus of claim 45, wherein each of said alignment grooves has a centerline substantially coplanar with the plane of said fiber array.

49. The apparatus of claim 45 wherein said channels are substantially parallel and evenly spaced.

50. The apparatus of claim 45, wherein said predetermined plane is substantially coplanar with a plane in which an acousto-optic modulator is able to deflect a free beam of radiation into each of said optical fibers.

51. The apparatus of claim 50, wherein said radiation beam has a radiation source comprising a laser.

52. The apparatus of claim 50, in which said radiation beam is to be delivered via said optical fibers to a mammalian body lumen for use in removing a portion of a total or partial occlusion from said lumen.

53. An apparatus for accurately positioning at least one optical fiber within a predetermined plane, comprising:
a connector comprising two plates, each plate having at least one groove in a surface, said plates integrally connected together with said grooved surfaces opposing one another, such that said grooves align to form at least one channel in said connector;
at least one optical fiber having a proximal end and a distal end, said proximal end of said fiber positioned within said at least one channel;
said connector further having at least two alignment grooves, one of said at least two alignment grooves positioned in each of two opposite surfaces of said connector, said alignment grooves for positioning said connector between two opposed rod-like structures so that said proximal end of said fiber is substantially within said predetermined plane.

54. The apparatus of claim 53, wherein each of said two opposed plates comprises silicon, and wherein each said groove in each plate is lithographically-etched.

55. The apparatus of claim 53, wherein each said channel groove has a substantially equal depth and wherein said opposed plates do not directly contact one another when said at least one fiber is positioned in said at least one channel.

56. The apparatus of claim 53, wherein said two rod-like structures are substantially parallel, and each of said structures has a longitudinal axis substantially coplanar with said predetermined plane.

57. The apparatus of claim 53, wherein each of said alignment grooves has a centerline substantially coplanar with said proximal end of said at least one fiber.

58. The apparatus of claim 53, wherein said predetermined plane is substantially coplanar with a plane in which an acousto-optic modulator is able to deflect a free beam of radiation into said at least one optical fiber.

59. The apparatus of claim 58, wherein said radiation beam has a radiation source comprising a laser.

60. The apparatus of claim 58, in which said radiation beam is to be delivered via said optical fiber to a mammalian body lumen for use in removing a portion of a total or partial occlusion from said lumen.

61. The apparatus of claim 53, wherein said plates are connected with glue and said glue and said channel cooperate to hold said fiber immovably in place.

62. A method of aligning a plurality of optical fibers within a predetermined plane, comprising:
immovably positioning said plurality of fibers within a substantially planar array, and
positioning said array relative to two opposed rod-like structures so that said fiber array is substantially coplanar with said predetermined plane.

63. The method of claim 62, wherein said predetermined plane is substantially coplanar with an operating plane of an acousto-optic modulator, said method further comprising deflecting a free beam of radiation into each of said optical fibers.

64. The method of claim 63, wherein said radiation beam comprises a laser beam.

65. The method of claim 63, further comprising delivering said radiation beam via at least one of said optical fibers to a mammalian body lumen, and using said radiation beam to remove a portion of a total or partial occlusion from said lumen.
